(19) United States
(12) Patent Application Publication
(10) Pub. No.: US 2024/0423816 A1
(43) Pub. Date: Dec. 26, 2024

(54) ORTHOPEDIC SYSTEM AND PROSTHETIC FOOT HAVING SUCH AN ORTHOPEDIC SYSTEM

(71) Applicant: OTTOBOCK SE & CO. KGAA, Duderstadt (DE)

(72) Inventors: Martin PUSCH, Duderstadt (DE); Hannes KRENZ, Duderstadt (DE); Georg GEHRMANN, Duderstadt (DE); Carsten MOENICKE, Duderstadt (DE); Nils BENSTEM, Duderstadt (DE)

(21) Appl. No.: 18/691,590

(22) PCT Filed: Sep. 29, 2022
(86) PCT No.: PCT/EP2022/077187

§ 371 Date: Mar. 13, 2024

(30) Foreign Application Priority Data
Sep. 30, 2021 (DE) 10 2021 125 385.3

(51) Int. Cl. A61F 2/66; A61F 2/50

(52) U.S. Cl. CPC A61F 2/66; A61F 2002/5009; A61F 2002/6664

(57) ABSTRACT

The invention relates to an orthopedic system having a leaf spring element (40) and a bearing component (600) supported on the main surface of the spring element and being fixable to the leaf spring element. At least one elastomeric element (71, 72) is arranged between the bearing component and the main surface of the leaf spring element and is fastened to the leaf spring element and the bearing component such that it transfers shearing forces.

The invention relates to an orthopedic system having a leaf spring element and a bearing component which is supported on a main surface of the leaf spring element and is fixed to the leaf spring element, and a prosthetic foot having such an orthopedic system.

Leaf spring elements are often used in orthopedic systems to store deformation energy and at the same time to provide force limitation. Modern leaf spring elements are made of fiber composite materials or have components made of fiber composite materials and are part of complex systems, for example prosthetic feet. To make effective use of the leaf spring elements, they have to be installed in holders in order to allow further components to be mounted on the leaf spring element. Holders or other components are fastened to a leaf spring element by form-fit engagement, for example, with the component that is to be mounted, for example a metal component, being screwed onto the leaf spring element. Such a solution is described in U.S. Pat. No. 10,390,974 B2 and EP 3 128 958 B1.

From WO 2020/152341 A1 it is known that a leaf spring element can be connected to a carrier via an adhesive connection, a clamping connection and/or a form-fit connection. In the case of form-fit connections via screws, holes are introduced into the leaf spring element, through which holes corresponding bolts or screws are then passed and thus connected to the further component. Holes weaken the leaf spring component and are therefore not to be applied everywhere. In particular, the mechanical properties of fiber composite materials are adversely affected by holes or faults in the fiber structure. Attachment parts cannot be mounted, or are difficult to mount, especially in regions of great deformation of the leaf spring elements.

The object of the present invention is therefore to make available an orthopedic system having a leaf spring element which permits greater freedom of design without limiting the durability and load-bearing capacity of the overall system.

According to the invention, this object is achieved by an orthopedic system having the features of the main claim and by a prosthetic foot having such an orthopedic system. Advantageous embodiments and further developments of the invention are disclosed in the description, the subclaims and the figures.

In the orthopedic system having a leaf spring element and a bearing component which is supported on a main surface of the leaf spring element and is fixed to the leaf spring element, provision is made that at least one elastomer element is arranged between the bearing component and the main surface of the leaf spring element and is fastened to the leaf spring element and to the bearing component in a manner transmitting tensile forces and shear forces. Between the bearing component and the leaf spring element, an elastic connection is thus present which permits a relative movement between the leaf spring element and the bearing component. It is thereby possible, despite a safe and load-bearing assignment of the bearing component to the leaf spring element, also to arrange and position the bearing component in regions of high deformation of the leaf spring element. The fact that the bearing component is fastened to the leaf spring element in such a way that tensile forces and shear forces, applied to the leaf spring element via the bearing component, can also be transmitted safely and permanently to the leaf spring element allows the bearing component to be placed at almost any desired position on the leaf spring element, regardless of the deformation situation of the leaf spring element during use of the orthopedic system. This increases the design freedom in the configuration of the orthopedic system, or of the overall system of which the orthopedic system is a part.

In one embodiment, the bearing component has at least one flat support surface, via which the elastomer element is supported on the bearing component and via which the connection to the leaf spring element is produced. The flat support surface allows forces to be introduced over a large area and avoids load peaks of forces that are introduced into the leaf spring element via the elastomer element from the usually rigid bearing component.

In a further development, the bearing component is provided with a plurality of support surfaces spaced apart from one another, such that the bearing component is mounted and fastened at a plurality of spaced-apart regions of the leaf spring element. The two support surfaces are connected to each other in a bridge-like manner by the bearing component and permit an optimized application of forces and an improved load distribution, so that the mechanical properties of the leaf spring element can be optimally exploited.

In one embodiment, the elastomer element is fastened to the leaf spring element via a pretensioning device. The pretensioning device can be formed, for example, as a bracket, a clamping system, a belt or a screw connection. As a result of the pretensioning by the pretensioning device, the three components of the system, i.e. the leaf spring element, the bearing component and the elastomer element, are pressed together, thereby avoiding a situation where the assignment of the components to one another is lost. Noise generated by components striking against one another is avoided and, in addition, the stable application of force gives the user of the orthopedic system a safe feeling.

In one embodiment, the at least one elastomer element is glued to the bearing component and/or the leaf spring element. When the elastomer element is glued to both the bearing component and the leaf spring element, a cohesively bonded coupling of all three elements of the system is guaranteed in a simple, cost-effective and durable manner. The structure of the leaf spring element is not adversely affected by the fastening of the bearing component. The same applies when the elastomer element is glued to the leaf spring element and it is coupled and fastened to the bearing component via another fastening device or a fastening element, for example by screws. It is also possible, by clamping with clamping devices, to fasten the leaf spring element to the bearing component with interposition of the elastomer element. Even if a screw connection is effected with a through-hole through the leaf spring element, the mechanical loading of a force transmission via the bearing component is reduced by the interpositioning of the elastomer element, and an advantage is afforded with regard to the design freedom and durability of the orthopedic system.

The bearing component can have at least one stop which, in a starting position where no forces are transmitted from the bearing component to the leaf spring element by the use of the orthopedic system, is arranged spaced apart from a secondary surface or a stop element of the leaf spring element. Leaf spring elements have a longitudinal extent and, in cross section with respect to the longitudinal extent, are substantially rectangular with two long sides and two mutually opposite short sides. The surface assigned to the long sides is a main surface, and the surface assigned to the short sides or narrow sides is a secondary surface. A stop or a plurality of stops can be arranged on the side of the leaf spring element. On account of the distance to the secondary surface, a mobility is possible transverse to the longitudinal extent of the leaf spring element. Compensation movements and displacement movements are made possible and absorbed by the elastomer element. The deformations of the elastomer element are permitted until the stop makes contact with the leaf spring element. A secondary surface can also be formed within the leaf spring element in a bore or in a slot. Thus, a stop arranged on or in the bearing component can engage in a slot, a hole or another recess, wherein without a load, in particular without a load in the main plane, the stop is not in direct contact with the leaf spring element. Alternatively or in addition to a direct contact with the leaf spring element, a stop element can be arranged or formed on the leaf spring element, which stop element strikes against a stop or a recess of the bearing component or comes into contact with it when a load limit is reached and the relative displacement of bearing component to leaf spring element is too great. The displacement can be a rotation or shifting of the bearing component with respect to the leaf spring element.

In one embodiment, a plurality of stops or stop elements are arranged or formed opposite one another. In particular, a plurality of stops are arranged laterally alongside the leaf spring element and/or the stop element and frame this or these from two or more sides. Abutment then preferably takes place on one of the secondary surfaces or on two stop elements on the leaf spring element.

Advantageously, the bearing component is mounted elastically on the leaf spring element in three rotational and three translational degrees of freedom, wherein, on account of the dimensions of the elastomer element, only slight rotations and/or shifts in the respective directions or about the respective axes are possible. In an advantageous embodiment, it is provided that a torsion in the transverse plane is in a range between +/−1° and +/−5°, in particular +/−2.5°. Due to the fact that there is no rigid fastening of the leaf spring element to the bearing component, a compliance under torsional loads is made possible, in particular in the frontal plane and the transverse plane, wherein the reference plane for this is the plane in which the main surface of the leaf spring element lies. The connection between the bearing component and the leaf spring element is designed in such a way that a relative movement between the two components is possible to a limited extent. At the same time, a strong deformation of the leaf spring element in all directions is possible to a limited extent, without direct collision between the leaf spring element and the bearing component. Nevertheless, high mechanical forces and loads are transmitted from the bearing component to the leaf spring element, wherein the transmission of normal forces from the leaf spring to the bearing block will be the main force transmission device.

Advantageously, the leaf spring element is produced from a fiber composite material, and the bearing component is in particular produced from a metal or a metal alloy, in particular a light metal alloy. The respective mechanical loads are thus optimally absorbed and transmitted by the respective material.

The bearing component is designed in particular as a bearing block with a bearing receptacle, so that an articulated connection of further components of an orthopedic overall system, in particular of a further prosthesis component, can take place. Instead of an articulated connection of further components to the bearing component, one or more components can be rigidly or resiliently fastened to the bearing component. A pyramid adapter can be directly fastened to or formed on the bearing component or can be coupled to the rest of the bearing component via an elastic component. The connection of other components to the bearing component, in particular a lateral and/or proximal connection, can also be rotationally fixed or rotationally rigid, which is to be understood as meaning both a one-piece configuration and a multi-part configuration.

In one embodiment, the bearing component is arranged in the middle third between the ends of the leaf spring element, as a result of which force is introduced in those regions of the leaf spring element that are particularly resilient, when the remaining bearing of the leaf spring element takes place at its ends. The introduction of force in regions with high deformation allows the spring properties of the leaf spring element to be efficiently exploited, and a fine response is obtained when forces are introduced from the bearing component into the leaf spring element via the elastomer element.

The elastomer element or the elastomer elements are preferably made of a permanently elastic material, in particular of a polyurethane elastomer, which advantageously has a Shore A hardness of between A40 and A80, preferably of between A50 and A70, and particularly preferably of between A55 and A65. It is also possible to use other elastomers that are permanently elastic and durable over the service life.

The invention relates in particular to a prosthetic foot insert having an orthopedic system as described above. With such a design, it is possible to reduce the loading of the leaf spring element by virtue of large-area load introduction over the entire width of the main side. Load peaks caused by a direct coupling of the bearing component to the leaf spring element are avoided. In addition, an increased flexibility of the prosthetic foot is obtained for the prosthesis user, since a rotational resilience of the prosthetic foot in the frontal plane results via the connection of the bearing component with interpositioning of at least one elastomer element. The use of the prosthetic foot when turning is also made easier, since there is rotational flexibility in the transverse plane.

Embodiments of the invention are explained in more detail below with reference to the figures. The same reference signs denote the same components. In the figures:

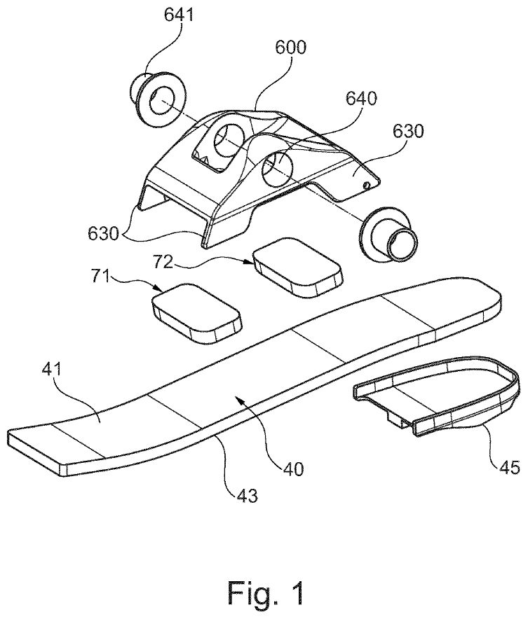

FIG. 1 shows an orthopedic system in an exploded view;

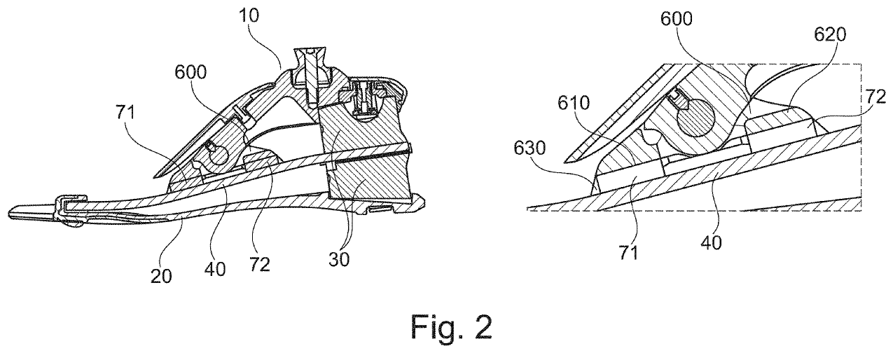

FIG. 2 shows schematic sectional views of a prosthetic foot;

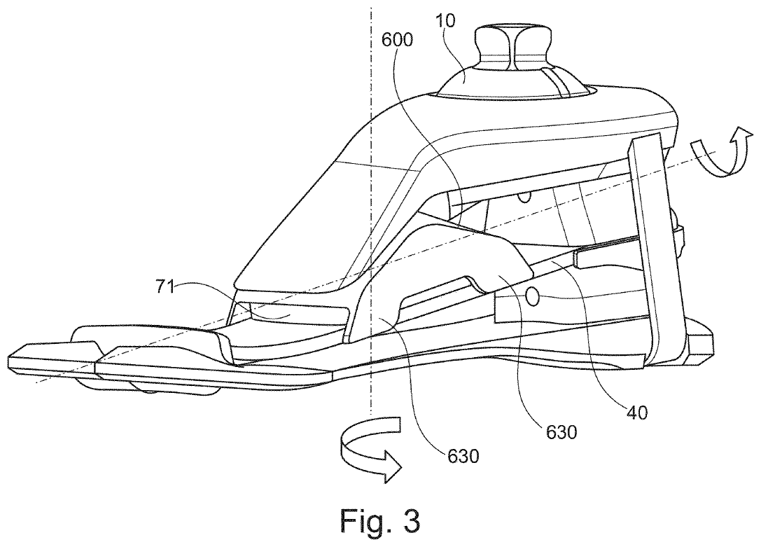

FIG. 3 shows a schematic overall view of a prosthetic foot;

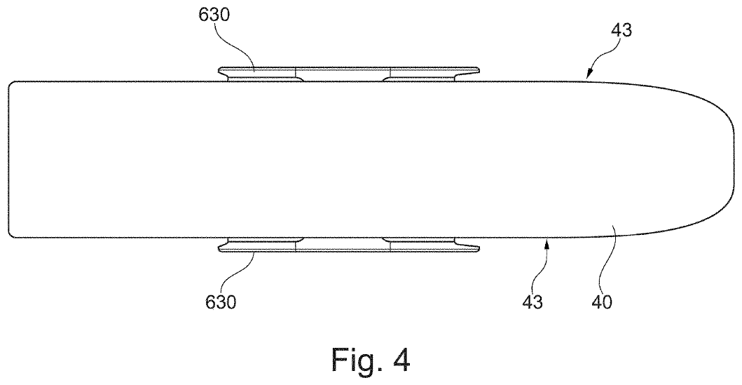

FIG. 4 shows a bottom view of an orthopedic system;

FIG. 5 shows a front view of an orthopedic system;

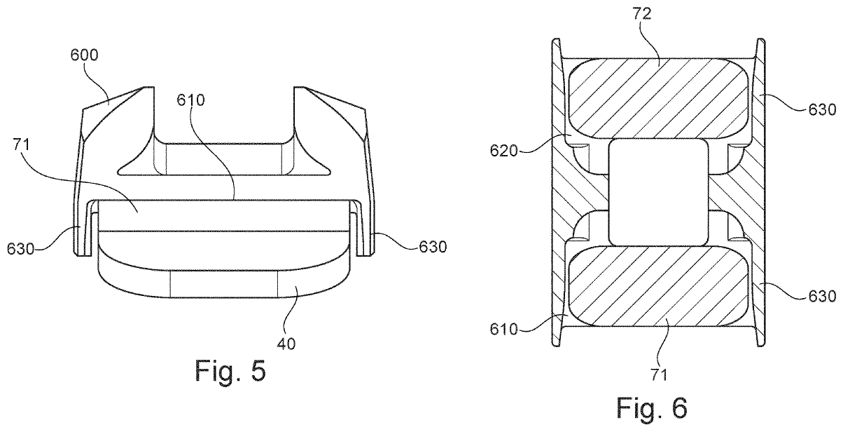

FIG. 6 shows a bottom view of a bearing component;

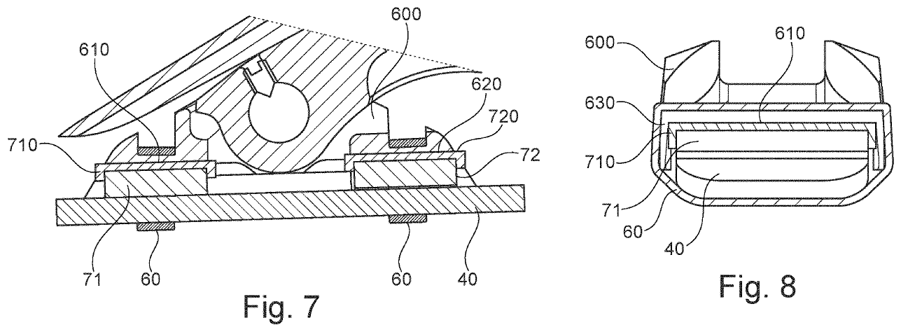

FIG. 7 shows an enlarged sectional view through a variant;

FIG. 8 shows a cross-sectional view through the variant according to FIG. 7; and

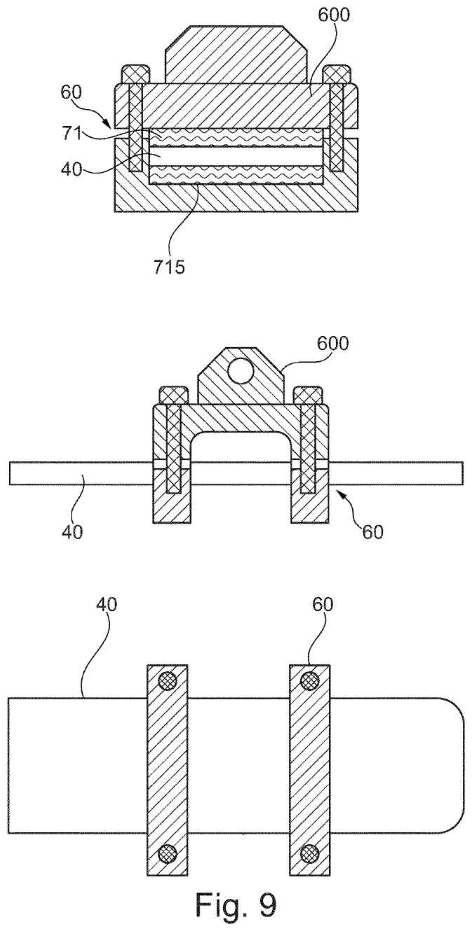

FIG. 9 shows three views of a clamped variant.

FIG. 1 shows an exploded view of an orthopedic system having a bearing component 600 in the form of a bearing block, which has a bearing receptacle 640 with associated bearing shells 641. An axle, around which the bearing component 600 can be pivoted, is introduced into the bearing receptacle 640. The bearing component 600 can be fastened pivotably to a carrier for example, which in turn has a proximal fastening device for fixing to a further prosthesis component, for example as part of a prosthetic foot, which is fastened to an ankle joint or to a lower-leg tube or a lower-leg socket. The bearing component 600 is constructed like a bridge and, on the underside, has two flat support surfaces, which are explained in more detail later.

A leaf spring element 40 made of a fiber composite material is shown below the bearing component 600. The leaf spring element 40 has a substantially rectangular cross section, with an upper face and a lower face as the main surface 41 and with two short side edges as secondary surfaces 43. The leaf spring element 40 is substantially rectilinear, although it may also have a slight curvature or an undulating shape. A heel element 45 can be fixed to the underside of the leaf spring element 40. As an alternative to the heel element 45, cushioning elements or coupling devices for attaching further components of a complete system can be fastened to the underside and/or upper face of the leaf spring element 40.

In the exemplary embodiment shown, two elastomer elements 71, 72 are arranged between the upper face on the main surface 41 of the leaf spring element 40 and the underside with the support surfaces of the bearing component 600, which elastomer elements 70, 72 lie flat on both the leaf spring element 40 and the bearing component 600. Stops 630, which are formed on the bearing component 600, are located laterally alongside the leaf spring element 40 and alongside the elastomer elements 71, 72. The stops 630 are downwardly protruding projections which ensure a lateral limitation of a displacement of the bearing component 600 relative to the leaf spring element 40. The elastomer elements 71, 72 are fastened, spaced apart from each other, to the leaf spring element 40 and to the bearing component 600, in particular by gluing or welding. Alternative types of fastening, for example an additional or alternative form-fit fastening, are provided, on condition that they allow transmission of shear forces and tensile forces to the respective elastomer elements 71, 72.

As an alternative to the total of four stops 630, it is also possible for just two stops 630 to be arranged or formed diagonally opposite each other or one behind the other on one side. A further alternative is that, instead of a lateral arrangement and a possible abutment of the stops 630 on the secondary surfaces 43, a stop element is arranged or formed on the surface of the leaf spring element 40 and is framed by two or more stops and initially allows a relative displacement of the leaf spring element 40 relative to the bearing component 600 and then limits this when a limit load is present. As a result, too great a deformation of the elastomer elements 71, 72 is limited, and the maximum movement from the bearing component 600 to the leaf spring element 40 is fixed. Such limitation of movement can also be achieved by forming in or introducing into the leaf spring element 40 a slit, a recess or an indent, in which a corresponding element, for example a peg, a tab or a pin, is inserted and, after overcoming elastic restoring forces of the elastomer elements 71, 72, comes into abutment with the leaf spring element 40.

FIG. 2 shows sectional views through a prosthetic foot having an orthopedic system as described above. The bearing component 600 described above can be seen, likewise the two elastomer elements 71, 72 and the leaf spring element 40, on the upper face of which the bearing component 600 is arranged with the stops 630. On the underside of the bearing component 600, the respective flat support surfaces 610, 620 are formed, which extend substantially parallel to the main surface 41 of the leaf spring element 40. In the exemplary embodiment shown, the elastomer elements 71, 72 are glued to both the leaf spring element 40 and the bearing component 600. In the heel region of the prosthetic foot, cushioning elements 30 are arranged on and fastened to the upper face and underside of the leaf spring element 40. The lower cushioning element 30 is supported on a base spring 20, which is coupled or connected in the forefoot region to the leaf spring element 40. The upper cushioning element 30 is supported on a carrier 10, on which in turn the bearing component 600 is mounted in the bearing receptacle, pivotably about the pivot axis. A pyramid adapter for fastening the prosthetic foot to a proximal prosthesis component is fastened to the upper face of the carrier 10.

FIG. 3 shows the prosthetic foot according to FIG. 2 in a perspective overall view. The carrier 10 is pretensioned with respect to the base spring 20 via a tensioning device and clamps the leaf spring element 40 between the two cushioning elements 30. The upper cushioning element 30 permits a slight movement of the carrier 10 relative to the leaf spring element 40 in the heel region or in the rear region. The elastic bearing and the arrangement of the stops 630 spaced apart from the secondary surfaces 43 of the leaf spring element 40 permit a controlled, slight rotation both about the axis perpendicular to the transverse plane and about an axis perpendicular to the frontal plane. In principle, rotation is also possible about an axis perpendicular to the sagittal plane, which runs substantially parallel to the axis of the bearing receptacle 640. However, on account of the pivotable mounting of the carrier 10 on the bearing component, uniform loading will occur on both the rear and the front elastomer element 71, 72, such that a rotation about this degree of freedom, although possible, is not relevant in practice. In addition to a rotation about three rotational degrees of freedom, the mounting via the elastomer elements 71, 72 also permits a movement in three translational degrees of freedom.

It will be seen from the bottom view shown in FIG. 4 that the secondary surfaces 43 of the spring element 40 are arranged spaced apart from the stops 630 of the bearing component 600, and therefore a rotation and a movement of the entire spring element 40 relative to the bearing component 600 is possible in all three rotational and all three translational degrees of freedom.

FIG. 5 shows a front view of the bearing component 600 with the front, flat contact surface 610, the two lateral stops 630, and the elastomer elements 71, 72 between the leaf spring element 40 and the contact surface 610. FIG. 6 shows a bottom view of the bearing component 600 with the two flat support surfaces 610, 620 for the two elastomer elements 71, 72 arranged spaced apart from each other. The stops 630 are positioned at a distance to the right and left or medially and laterally with respect to the lateral edges of the elastomer elements 71, 72. The elastomer elements 71, 72 extend over the entire width of the leaf spring element 40 and thus permit large-area introduction of forces uniformly, on account of the elastic properties, into the leaf spring element 40, across the full width of the latter. By virtue of the large-area introduction of forces, load peaks on the leaf spring element 40 are avoided, the mechanical load is reduced, and damage is prevented. The bearing component 600 is constructed like a bridge, as a result of which a clearance or a gap is formed between the two elastomer elements 71, 72, and therefore, with central loading via the bearing receptacle 640, the introduction of force along the longitudinal extent of the leaf spring element 40 is divided into two regions or surfaces spaced apart from each other.

FIGS. 7 and 8 show variants of the invention in which, respectively, two elastomer elements 71, 710 and 72, 720 are arranged between the respective support surfaces 610, 620 and the leaf spring element 40. The respective elastomer elements 71, 710 and 72, 720 can be fastened in different ways to the respective component, that is to the bearing component 600 and the leaf spring element 40. In one exemplary embodiment, it is provided that the elastomer element 710, 720 arranged on the contact surface of the bearing component 600 is fastened to the bearing component 600 by form-fit engagement, while the other elastomer element 71, 72 is glued to the surface of the leaf spring element 40. The upper elastomer element 710, 720 can have side walls or a recess into which the lower elastomer element 71, 72 can be inserted or applied, such that a movement and/or rotation of the elastomer elements 71, 710, 72, 720 with respect to one another is not possible or is possible only in certain directions. Depending on the shape of the elastomer elements, it may be possible to provide rotation about an axis perpendicular to the transverse plane. Rotation and tilting of the bearing component 600 relative to the leaf spring element 40 is still possible on account of the elastic properties of the elastomer elements 71, 710, 72, 720. There is also the possibility of different types of fastening being applied at different positions, such that, for example, the upper elastomer element 710 is glued in the front region of the bearing component 600 and the lower elastomer element 71 is fastened by form-fit engagement or fixed to the leaf spring element 40 via a clamping device. In principle, it is also possible for both elastomer elements 71, 710 to be glued to the respective component. The arrangement of a plurality of elastomer elements 71, 710; 72, 720 between the bearing component 600 and the leaf spring element 40 increases the design freedom in the provision of the elastic properties. With an exchangeable fastening of an elastomer element, it is easier to make adaptations to the respective preferences of the respective user. For example, if a user wants less flexibility at one or another location of the orthopedic system or of the prosthetic foot, this can be achieved, for example, by exchanging the elastomer elements 710, 720 fastened to the bearing component 600 via a screw connection. By virtue of the bearing component 600 preferably being made from metal, a screw connection of the elastomer elements 710, 720 to the bearing component 600 is readily possible without any significant structural impairment of the strength properties or mechanical properties.

In the exemplary embodiment shown, in order to prevent separation between the elastomer elements 71, 710; 72, 720 when said elastomer elements 71, 710; 72, 210 are not fixed to each other, pretensioning devices 60 in the form of belts are arranged both on the bearing component 600 and on the leaf spring element 40. The pretensioning devices 60 can be flexible and rigid under tension or elastic. They are guided within guides on the bearing component 600 and below the leaf spring element 40 and cause a pretensioning of the bearing component 600 in the direction of the leaf spring element 40. As a result, the elastomer elements 71, 710, 72, 720 arranged in pairs are pressed against each other and held permanently together. The pretensioning device 60 can also be designed to be releasable, so that, for exchanging elastomer elements 71, 710, 72, 720, the pretensioning device 60 is released in each case and then applied again. The pretensioning device 60 can also be designed differently, for example as a screw connection.

It will be seen from FIG. 8 that the lateral stops 630 are spaced apart both from the leaf spring element 40 and from the lower elastomer element 71, which is fastened to the leaf spring element 40. The upper elastomer element 710 arranged on the bearing component 600 fills the distance or clearance between the inner side of the stop 630 and the lower elastomer element 71, such that additional lateral guiding is provided. In addition to bridging of this distance, the side walls of the upper elastomer element 710 can also leave free a clearance from the inner surfaces of the respective stop 630 or only one stop 630. Depending on the design of the elastomer elements, it is possible to influence the movement behavior of the bearing component 600 relative to the leaf spring element 40.

FIG. 9 shows three views of a further variant, in which the leaf spring element 40 is embedded between two elastomer elements 71, 715. An elastomer element 71 is arranged between the support surface of bearing component 600 and the upper face of the leaf spring element 40, while the other elastomer element 715 is arranged below the leaf spring element 40 and supports the latter against a pretensioning device 60 which, in the exemplary embodiment shown, is formed as a clamping bracket, which is fixed releasably to the bearing component 600 via two screws. The clamping bracket extends below the leaf spring element 40 and is U-shaped. Internal threads for receiving the screws are arranged in the two upwardly extending legs of the clamping bracket. The leaf spring element 40 is guided laterally through the two upwardly projecting legs, wherein a lateral spacing may exist between the inner faces of the upwardly projecting legs and the leaf spring element 40, so as to permit a rotation and displacement relative to the bearing component 600. The two screws are guided through through-holes in the bearing component 600; by loosening or tightening of the screws, the pretensioning of the elastomer components 71, 715 can be individually varied. The view at the foot of FIG. 9 is a bottom view of the orthopedic system as part of a prosthetic foot, from which it can be seen that the respective clamping bracket extends over the entire width below the leaf spring element 40. Here too, two pretensioning devices 60 spaced apart from each other in the longitudinal extent of the leaf spring element 40 are arranged on the bearing component 600 and allow force to be introduced into two spaced apart regions of the leaf spring element 40. The pretensioning can be adjusted continuously. In addition to a U-shaped configuration of the clamping bracket, the pretensioning device 60 can also be formed simply as a strip-shaped plate. Instead of a screw, other force transmission devices may also be part of the pretensioning device 60 in order to pretension the leaf spring element 40 relative to the bearing component 600, with at least one elastomer element 71 arranged between them, and to clamp them together.

The leaf spring element is advantageously supported at its two end regions on other components or on the ground, so that in total a four-point bearing is obtained, with a bearing at the two end regions and with introduction of force at two spaced-apart force introduction regions provided between the bearing points at the end regions. In an embodiment of the bearing component 600 as a bearing block with a bearing receptacle 640 between two support surfaces 610, 620, it is advantageous to position the bearing receptacle 640 on the leaf spring element 40 in the middle of the spring length or within the middle fifth of the leaf spring element 40. This ensures uniform loading of the leaf spring element 40. Relative to the total length of the prosthetic foot insert, the positioning of the bearing receptacle 640 is shifted slightly further in the anterior direction on account of the greater total length of the base spring 20 compared to the leaf spring element 40 and the non-central arrangement of the leaf spring element 40 above the base spring 20. In a fully assembled prosthetic foot with a cosmetic cover, the bearing receptacle 640 is located slightly behind the center, in relation to the total length, but still within the middle fifth of the total length of the prosthetic foot.

1. An orthopedic system, comprising
a leaf spring element having a main surface;
a bearing component supported on the main surface of the leaf spring element, wherein the bearing component is fixed to the leaf spring element; and
at least one elastomer element arranged between the bearing component and the main surface of the leaf spring element, wherein the at least one elastomer element is fastened to the leaf spring element and to the bearing component in a manner configured for transmitting tensile forces and shear forces.

2. The orthopedic system as claimed in claim 1, wherein the bearing component comprises at least one flat support surface.

3. The orthopedic system as claimed in claim 1 wherein the bearing component comprises a plurality of support surfaces spaced apart from one another.

4. The orthopedic system as claimed in claim 1 wherein the at least one elastomer element is fastened to the leaf spring element and/or the bearing component via a pretensioning device.

5. The orthopedic system as claimed in claim 1 wherein the at least one elastomer element is glued to the bearing component and/or the leaf spring element.

6. The orthopedic system as claimed in claim 1 further comprising at least one stop arranged on the bearing component, wherein the at least one stop is arranged, in a starting position, spaced apart from a secondary surface of the leaf spring element and/or a leaf spring stop element of the leaf spring element.

7. The orthopedic system as claimed in claim 6, wherein the at least one stop comprises a plurality of stops arranged opposite one another.

8. The orthopedic system as claimed in claim 1 wherein the bearing component is mounted elastically on the leaf spring element in three rotational and translational degrees of freedom.

9. The orthopedic system as claimed in claim 1 wherein the leaf spring element comprises a fiber composite material and the bearing component comprises a metal.

10. The orthopedic system as claimed in claim 1 wherein the bearing component is formed as a bearing block with a bearing receptacle.

11. The orthopedic system as claimed in claim 1 further comprising a component is fastened to or formed on the bearing component in a rotationally rigid or elastic manner.

12. The orthopedic system as claimed in claim 1 wherein the bearing component is arranged in a middle third between a first end and a second end of the leaf spring element.

13. A prosthetic foot insert having an orthopedic system as claimed in claim 1.
